(19) United States
(12) Patent Application Publication
(10) Pub. No.: US 2011/0004311 A1
(43) Pub. Date: Jan. 6, 2011

(54) SHAPED IMPLANTS FOR TISSUE REPAIR

(76) Inventors: Eric J. Semler, Piscataway, NJ (US); Judith I. Yannariello-Brown, Somerset, NJ (US); Morris L. Jacobs, Newtown, PA (US)

Correspondence Address: GREENBERG TRAURIG, LLP, 200 PARK AVE., P.O. BOX 677, FLORHAM PARK, NJ, 07932, US

(21) Appl. No.: 12/814,136

(22) Filed: Jun. 11, 2010

Related U.S. Application Data
(60) Provisional application No. 61/186,166, filed on Jun. 11, 2009.

(51) Int. Cl. A61F 2/44

(52) U.S. Cl. 623/17.16

(57) ABSTRACT

Shaped constructs for repair of a defect in a body part or tissue of a subject are discussed herein. More specifically, implants suitable for delivery to a subject, such as a subject having a defect in a body part or body tissue, are discussed. Even more specifically, implants for intervertebral disc repair comprising corticocancellous bone, which can extend into the nucleus pulposus of an intervertebral disc and can be integrally attached to the annulus fibrosus of the disc to keep the implant in position, are described. Also, implants for meniscal repair comprising corticocancellous bone, which can extend from the outer edge of the meniscus to the inner region of the meniscus and can be integrally attached to the meniscal rim to keep the implant in position, are described. Implants for the repair of defects in bone, cartilage, and fibrocartilage are further described. Further described are methods for making such implants and for delivering the implants to a defect in a body part or body tissue of a subject.

### CROSS-REFERENCE TO RELATED APPLICATION

The present application claims priority to U.S. Provisional Patent Application Ser. No. 61/186,166 filed Jun. 11, 2009, the entire disclosure of which is incorporated by reference herein in its entirety.

### FIELD OF THE INVENTION

Shaped constructs for repair of a defect in a body part or tissue of a subject are discussed herein. More specifically, implants suitable for delivery to a subject, such as a subject having a defect in a body part or body tissue, are discussed. Even more specifically, implants for intervertebral disc repair comprising corticocancellous bone, which can extend into the nucleus pulposus of an intervertebral disc and can be integrally attached to the annulus fibrosus of the disc to keep the implant in position, are described. Also, implants for meniscal repair comprising corticocancellous bone, which can extend from the outer edge of the meniscus to the inner region of the meniscus and can be integrally attached to the meniscal rim to keep the implant in position, are described. Implants for the repair of defects in bone, cartilage, and fibrocartilage are further described. Further described are methods for making such implants and for delivering the implants to a defect in a body part or body tissue of a subject.

### BACKGROUND OF THE INVENTION

An intervertebral or spinal disc primarily serves as a mechanical cushion between the vertebral bones, permitting a range of multi-axial motions within vertebral segments of the axial skeleton. The normal disc is a unique structure comprised of three component tissues: the nucleus pulposus (“NP”), the annulus fibrosus (“AF”), and two opposing vertebral end plates. The disc is connected to the adjacent superior and inferior vertebrae through the hyaline cartilage-based end plates. These end plates are each composed of thin cartilage overlying a thin layer of hard, cortical bone which attaches to the spongy, richly vascular, cancellous bone of the vertebral body.

The AF is a tough collagenous fibrocartilage annular ring which consists mainly of type I collagen fibers organized into many crisscrossed layers forming a tough, outer fibrous ring that binds together adjacent vertebrae. This fibrous portion, which is shaped much like a laminated automobile tire, consists of overlapping multiple plies at roughly a 30-degree angle in both directions.

The AF contains a complex, flexible, and hydrophilic core, the nucleus pulposus (NP). The NP consists of a hydrogel-like composite made of proteoglycan and type II collagen that releases water rapidly when load is applied to the spine (sitting up, standing, hip rotation, walking, etc.). A healthy NP is largely a gel-like substance having a high water content, and, similar to air in a tire, serves to keep the AF tight yet flexible. The NP is connected to the AF, and the transition between these two bodies is gradual. The nucleus and the inner portion of the annulus have no direct blood supply. The natural physiology of the NP promotes fluids being brought into, and released from, the NP during cyclic loading. The ability of the NP to convert compressive forces in the spine into tensile forces on the AF may keep proper tension in the AF and prevent the AF from weakening.

The spinal disc may degenerate or be displaced or damaged due to trauma or a disease process. A disc herniation occurs when the fibers of the AF are weakened or torn and the inner tissue of the NP becomes permanently bulged, distended, or extruded out of its normal, internal annular confines. The mass of a herniated or “slipped” nucleus can compress a spinal nerve, resulting in leg pain, loss of muscle control, or even paralysis. Alternatively, with disc degeneration, the NP loses its water binding ability and loses volume, as though the air had been let out of a tire. Subsequently, the height of the NP decreases, leading to inadequate tension on the AF. Over time, these events may cause the AF to buckle in areas where the laminated plies are loosely bonded. As these overlapping laminated plies of the AF begin to buckle and separate, either circumferential or radial annular tears may occur, which can result in renewed impingement by the AF on nerve structures posterior to the disc. These events may lead to instability of the disc with increased loading on the AF. The partially enervated AF may become painful under these conditions and cause persistent and disabling lower back pain. Furthermore, loss of disc height resulting from loss of NP integrity may increase loading on the facet joints. Adjacent, ancillary spinal facet joints will be forced into an overriding position, which can result in deterioration of facet cartilage and, ultimately, osteoarthritis and additional back pain. As the joint space decreases, the neural foramina formed by the inferior and superior vertebral pedicles close down. This leads to foraminal stenosis, pinching of the traversing nerve root, and recurring radicular pain. The most common resulting symptoms of disc degeneration are pain radiating along a compressed nerve and low back pain, both of which can be crippling for the patient. The significance of this problem is increased by the low average age of diagnosis, with over 80% of disc degeneration patients in the U.S. being under 59.

Current surgical solutions for treating intervertebral disc herniation include removing the disc material impinging on the nerve roots or spinal cord external to the disc by gripping and evulsing it off the AF. This is referred to as partial discectomy. Whenever the NP tissue is herniated or removed by surgery, the disc space will narrow and may lose much of its normal stability. In many cases, to alleviate pain from degenerated or herniated discs, a significant amount of disc material is removed and the two adjacent vertebrae are surgically fused together. While this treatment alleviates the pain, all disc motion is lost in the fused segment.

Surgical interventions, whether discectomy or fusion of adjacent vertebrae, generally lower the functionality of the spine in some way. For that reason, prosthetics for the spinal disc or its parts have been developed. The first prostheses embodied a wide variety of ideas primarily using mechanical devices and were designed to replace the entire intervertebral disc space. Since mechanical discs are large and require a significantly invasive procedure to implant, there are substantial risks during surgery and in cases where revision is necessary. Thus, in situations where the AF is still competent, it may be desirable to attempt to augment or replace only the NP portion of the disc with a less invasive surgical intervention. With respect to prostheses for replacing the NP, it has become apparent that an important feature of a prosthetic NP is that the AF is not entirely removed upon implantation. Normally, however, an opening of some type must be created through the AF. The prosthetic NP is then passed through this opening for implantation into the NP cavity. Because creation of this opening traumatizes the AF, it is highly desirable to minimize its size. Many prosthetic NP devices currently available do not account for this generally accepted implantation technique. For example, a relatively rigid prosthesis configured to approximate the shape of a natural NP requires an extremely large opening in the AF in order for the prosthetic device to “pass” into the NP cavity.

Accordingly, there is a need for additional implants and methods for treating spinal disc injuries.

Similar to an intervertebral disc, the meniscus is a specialized tissue found beneath the bones of a joint, functioning to add joint stability, distribute body weight across the joint, provide shock absorption, and deliver lubrication and nutrition to the joint. Menisci are made up of tough cartilage and conform to the surfaces of the bones upon which they rest. In the knee, for example, the meniscus is a C-shaped piece of fibrocartilage located at the peripheral aspect of the joint between the tibia and femur. The peripheral rim of the meniscus at the menisco-synovial junction is highly vascular, while the inner two-thirds portion of the meniscus is completely avascular, with a small transition between the two.

The meniscus may be injured or torn as a result of traumatic injuries such as a fall or athletic overexertion. For instance, the most common tear to the meniscus occurs when the knee joint is bent and the knee is then twisted. In addition, the meniscus begins to deteriorate with age, often developing degenerative tears. Meniscal tears can occur in either the thick outer part of the meniscus or through the thin inner portion, with some tears affecting only a small part of the meniscus and others affecting nearly the entire meniscus. Typically, when the meniscus is damaged, the torn piece begins to move in an abnormal fashion inside the joint. Because the space between the bones of the joint is very small, as the abnormally mobile meniscal tissue moves, it may become caught between the bones of the joint (i.e., the femur and tibia). When this happens, the knee becomes painful, swollen, and difficult to move.

A damaged meniscus is unable to undergo the normal healing process that occurs in other parts of the body due to the fact that, as mentioned above, the majority of the meniscus has no blood supply. Degenerative or traumatic tears to the meniscus which result in partial or complete loss of meniscal function frequently occur in the inner avascular portion, where the tissue has little potential for regeneration. Such tears generally result in severe joint pain and locking in the short term, as well as loss of meniscal function leading to arthritis in the long term.

Currently available treatments for meniscal injuries provide little opportunity for meniscal repair or regeneration. Meniscal tears that can be stabilized in vascularized areas of the meniscus can be repaired via suture or equivalent meniscal repair devices. Such repairs are successful in approximately 60-80% of cases. However, the percentage of injuries which meet the criteria to be repaired (i.e., vascularity, type of tear, stability and integrity of the meniscus, stability of the knee, and patient factors such as age and activity) in such a manner is very low (i.e., only 15% or less). Often the meniscal tear is in an avascular region of the meniscus, and thus, will not heal even if repaired. Moreover, some meniscal tears are frayed and cannot be sutured together. In the event that a meniscal repair is possible and the repair actually fails, then the next course of treatment is either a partial or total meniscectomy.

Most meniscal injuries are currently treated by removing the unstable tissue during a partial meniscectomy. Once the tissue is removed, however, no further treatment is conducted, and the patient is left with an abnormal meniscus. While most patients respond well to this treatment in the short term, they often develop degenerative joint disease several years post-operatively, with the amount of tissue removed potentially playing a part in the extent and speed of degeneration. When a majority of the meniscal tissue is injured, a total meniscectomy is performed in lieu of a partial meniscectomy, and if the patient continues to experience pain after a total meniscectomy without significant joint degeneration, a secondary treatment of meniscal allografts is possible. Accordingly, it may be desirable to utilize a device capable of repairing a majority of meniscal tears or injuries, either before or after a partial meniscectomy, in the long-term.

Accordingly, there is a need for additional implants and methods for treating injuries to the meniscus.

### SUMMARY OF THE INVENTION

Broadly speaking, the present invention involves an implant for the repair of tissue such as annulus fibrosus or meniscus. The implant has a plug section which is made of cancellous bone and is insertable in the defect. The implant also has at least one cap member made of cortical bone. The cap member is attached to the plug section so as to inhibit movement of the implant in the defect. The plug section and/or the cap member may be non-osteoinductive.

In certain embodiments, the implants described herein comprise a first cortical bone layer with a first thickness and a first dimension perpendicular to the first thickness, as well as a cancellous bone section having a top, a bottom, a second thickness, and a second dimension perpendicular to the second thickness. The bottom of the cancellous bone section is in contact with the first cortical bone layer in these embodiments. At least one of the first cortical bone layer and the cancellous bone section is at least partially demineralized. The first cortical bone layer may be integral with the cancellous bone section. In some embodiments, the first dimension may be the length of the first cortical bone layer, and the second dimension may be the length of the cancellous bone section. In such embodiments, the length of the cancellous bone section may be less than the length of the first cortical bone layer. In certain embodiments, the second thickness of the cancellous bone section may be greater than the first thickness of the first cortical bone layer. In particular embodiments, the implants further comprise a second cortical bone layer having a third thickness and a third dimension perpendicular to the third thickness. In these embodiments, the top of the cancellous bone section is in contact with the second cortical bone layer, and the first cortical bone layer may be substantially parallel to the second cortical bone layer.

The first and/or second cortical bone layer(s) and the cancellous bone section may have a variety of shapes, including, but not limited to, rectangular, cuboidal, discoid, cylindrical, ovoid, cup-shaped, conical, slanted cone, or dumbbell-shaped. In certain embodiments, the implant will resemble or have the shape of a three dimensional bandage or a three dimensional H-shaped bandage. Moreover, in some embodiments, the bone of the implant is obtained from the ilium, scapula, femur, tibia, humerus, talus, calcaneus, or patella of a mammal.

Furthermore, in certain embodiments, the first dimension is the length of the first cortical bone layer, and the first cortical bone layer comprises a plurality of collagen fibers that are oriented along the length of the cortical bone layer. In other embodiments, the first cortical bone layer further comprises a second dimension that is the width of the first cortical bone layer, and in these embodiments, a plurality of collagen fibers are oriented along with width of the first cortical bone layer. In other embodiments, the first dimension is the length of the first cortical bone layer and the first cortical bone layer comprises a plurality of collagen fibers are oriented at an angle to the length of the first cortical bone layer.

As discussed herein, in some embodiments, the implants described herein comprise at least one portion comprising partially demineralized, surface demineralized, or fully demineralized bone. In some embodiments, the portion of the implant comprising partially demineralized, surface demineralized, or fully demineralized bone is the first and/or second cortical bone layer. In other embodiments, the portion of the implant comprising partially demineralized, surface demineralized, or fully demineralized bone is the cancellous bone section. In yet other embodiments, both the cortical bone layer(s) and the cancellous bone section comprise partially demineralized, surface demineralized, or fully demineralized bone. In embodiments in which a portion or portions of the implant are demineralized, the demineralization may cause this portion or portions to have shape-memory. In additional embodiments, the implants described herein comprise bone that is non-osteoinductive or is treated to be non-osteoinductive. In other embodiments, the implants described herein comprise bone that has reduced osteoinductivity or is treated to have reduced osteoinductivity. The implants described herein may also further comprise cells or a growth factor(s).

Furthermore, in some embodiments, methods for making the implants described herein are described. In some embodiments, a method for making the implant comprises obtaining a section of corticocancellous bone comprising a first layer of cortical bone, a layer of cancellous bone that is integral with the first layer of cortical bone, and a second layer of cortical bone that is integral with the layer of cancellous bone; removing the second layer of cortical bone, and then shaping the first layer of cortical bone and the layer of cancellous bone to form the implant. In these embodiments, the implant comprises a first cortical bone layer having a first thickness and a first dimension perpendicular to the first thickness, and a cancellous bone section having a top, a bottom, a second thickness, and a second dimension perpendicular to the second thickness. In these embodiments, a portion or portions of the corticocancellous bone may be at least partially demineralized, and the demineralization may cause this portion or portions to have shape-memory. The corticocancellous bone may also be treated to render it non-osteoinductive or to reduce its osteoinductivity.

In other embodiments, a method for making the implant described herein comprises obtaining a section of corticocancellous bone comprising a first layer of cortical bone, a layer of cancellous bone that is integral with the first layer of cortical bone, and a second layer of cortical bone that is integral with the layer of cancellous bone; and shaping the first or second layer of cortical bone and the layer of cancellous bone to form the implant. In these embodiments, the implant comprises a first cortical bone layer having a first thickness and a first dimension perpendicular to the first thickness; a second cortical bone layer having a second thickness and a second dimension perpendicular to the second thickness; and a cancellous bone having a top, a bottom, a third thickness, and a third dimension perpendicular to the third thickness. In these embodiments, the cancellous bone section is integral with the first and second cortical bone layers of the implant. Also, the first cortical bone layer of the implant is in contact with the bottom of the cancellous bone section, and the second cortical bone layer is in contact with the top of the cancellous bone section. A portion or portions of the corticocancellous bone may be at least partially demineralized, and the demineralization may cause this portion or portions to have shape-memory. The corticocancellous bone may also be treated to render it non-osteoinductive or to reduce its osteoinductivity.

In yet other embodiments, a method for making the implant described herein comprises obtaining a first portion of cortical bone, obtaining a second portion of cancellous bone, and then shaping the first portion of cortical bone to form a cortical bone layer having a first thickness and a first dimension perpendicular to the first thickness, and shaping the second portion of cancellous bone to form a cancellous bone section having a top, a bottom, a second thickness, and a second dimension perpendicular to the second thickness. The cancellous bone section is then attached to the cortical bone layer such that the cancellous bone section is disposed on the cortical bone layer to form the implant and the cortical bone layer of the implant is in contact with the bottom of the cancellous bone section.

In further embodiments, methods for delivering an implant to a defect in a body part of a subject are described. In some embodiments, the method of delivering the implant comprises obtaining an implant comprising a first cortical bone layer having a first thickness and a first dimension perpendicular to the first thickness and a cancellous bone section having a top, a bottom, a second thickness, and a second dimension perpendicular to the second thickness, wherein the bottom of the cancellous bone section is in contact with the first cortical bone layer. In other embodiments, the implant may also comprise a second cortical bone layer having a third thickness and a third dimension perpendicular to the third thickness, wherein the top of the cancellous bone section is in contact with the second cortical bone layer, and wherein the first cortical bone layer is substantially parallel to the second cortical bone layer. In some embodiments, the implant is attached to a tissue of the subject, wherein the tissue has a height and a width. In certain embodiments, the implant may be attached to the tissue by suturing, stapling, or with a biological glue or adhesive. In some embodiments, the implant may already have sutures in it so that a surgeon implanting it would not have to thread the sutures in his or herself. In some embodiments, however, no additional means of attachment will be necessary. For example, in one embodiment, the shape of the implant itself may act as an anchor, holding the implant in place.

In some embodiments, the defect to which the implant is delivered may be in the intervertebral or spinal disc of a subject, while in other embodiments, the defect may be in the meniscus, the cartilage, the fibrocartilage, or the bone. Where used for spinal disc repair, the methods described herein comprise an implant that is attached to the annulus fibrosis of the subject. Where used for meniscal repair, the methods described herein comprise an implant that is attached to the meniscus of the subject. In yet other embodiments, the implants are used for prophylactic or preventative purposes.

### BRIEF DESCRIPTION OF THE DRAWINGS

For a better understanding of the present invention, reference is made to the following detailed description of an exemplary embodiment considered in conjunction with the accompanying drawings, in which:

FIGS. 1A-1B are perspective views of embodiments of an implant;

FIGS. 2A-2E show other embodiments of implants in which the cortical bone layer and the cancellous bone section have various shapes;

FIGS. 3A-3C show one embodiment of a method of making an implant;

FIGS. 4A-4E show another embodiment of a method of making an implant;

FIGS. 5A and 5B show an intervertebral disc and an intervertebral disc having a defect, respectively;

FIGS. 6A-6H show embodiments of an implant inserted into an intervertebral disc;

FIGS. 7A-7B show additional embodiments of implants inserted into an intervertebral disc;

FIG. 8 is a superior (top) view of a right knee, showing a lateral meniscus and a medial meniscus;

FIG. 9A shows a circumferential tear in a meniscus;

FIGS. 9B-9C show embodiments of implants in which the cortical bone layer and the cancellous bone section have various additional shapes;

FIG. 9D shows an embodiment of an implant inserted into a meniscus having a defect after the defective tissue is removed;

FIG. 10A shows a radial tear in a meniscus;

FIG. 10B shows an embodiment of an implant in which the cortical bone layer and the cancellous bone section have specific shapes; and

FIG. 10C shows an embodiment of an implant inserted into a meniscus having a defect after the defective tissue is removed.

### DETAILED DESCRIPTION OF THE EXEMPLARY EMBODIMENT

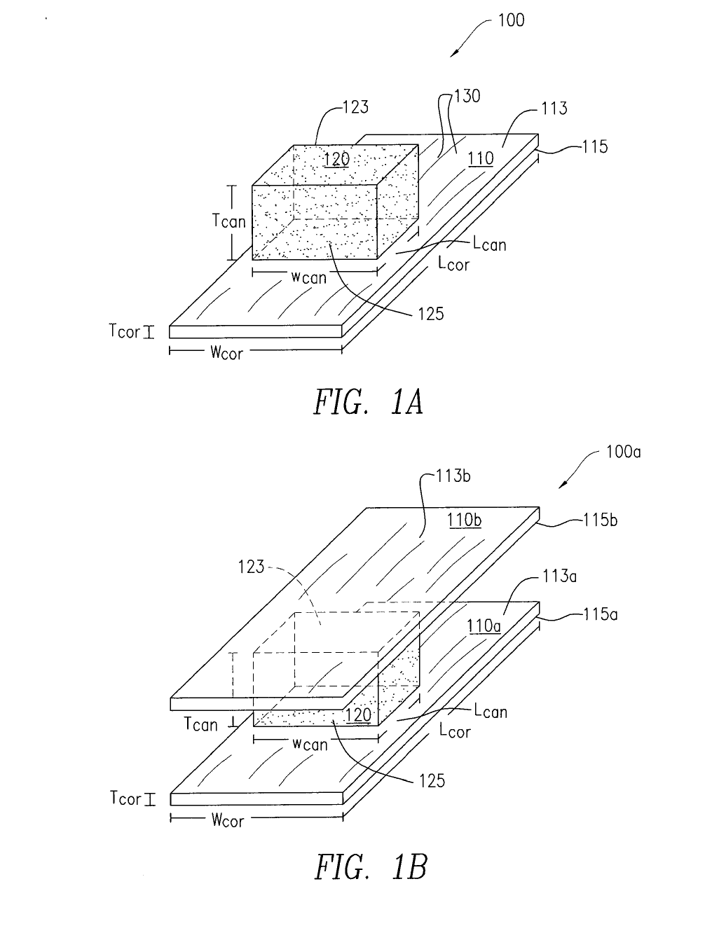

In some embodiments, an implant suitable for delivery to a subject, such as a subject having a defect in a body part or body tissue, comprises a cortical bone layer in contact with a cancellous bone section. FIG. 1A shows a perspective view of one such embodiment. In particular, FIG. 1A shows an implant 100 that comprises a cortical bone layer 110 (i.e., a cap member) and a cancellous bone section 120 (i.e., a plug section) that is in contact with the cortical bone layer 110. The cancellous bone section 120 has a top 123 and a bottom 125. The cortical bone layer 110 has a top 113 and a bottom 115. In this embodiment, the bottom 125 of the cancellous bone section 120 contacts the cortical bone layer 110. Also in this embodiment, the top 113 of the cortical bone layer 110 contacts the cancellous bone section 120. In other embodiments, the top 123 of the cancellous bone section 120 contacts the cortical bone layer 110. In yet other embodiments, the bottom 115 of the cortical bone layer 110 contacts the cancellous bone section 120. The cortical bone layer 110 can be integral with the cancellous bone section 120. For example, as discussed further below, the cortical bone layer and the cancellous bone section can be formed from corticocancellous bone in which the cortical bone is integral with the cancellous bone. Alternatively, the cortical bone layer can be attached or affixed to the cancellous bone section.

As shown in FIG. 1A, the cortical bone layer 110 has a thickness or height T_{cor}, a dimension that is perpendicular to the thickness, such as the length L_{cor}, and another dimension that is perpendicular to the thickness, such as the width W_{cor}. The thickness T_{cor} of the cortical bone layer 110 can range from about 0.1 mm to about 5.0 mm; about 0.25 mm to about 3.75 mm; about 0.5 mm to about 3.0 mm; or about 0.7 mm to about 2.6 mm. The other dimensions of the cortical bone layer 110, such as the length L_{cor} or width W_{cor} can be equal to or greater than about 2.0 mm; about 3.5 mm; about 5.0 mm; about 10.0 mm; about 15.0 mm; about 20.0 mm; about 25.0 mm; or about 30.0 mm.

The cancellous bone section 120 has a thickness or height T_{can}, a dimension that is perpendicular to the thickness of the cancellous bone section, such as the length L_{can}, and a another dimension that is perpendicular to the thickness of the cancellous bone section, such as the width W_{can}. The thickness T_{can} of the cancellous bone section 120 can range from about 2.0 mm to about 20.0 mm; about 3.5 mm to about 15.0 mm, or about 5.0 mm to about 10.0 mm. The thickness T_{can} may be equal to or greater than about 2.0 mm; about 3.5 mm; about 5.0 mm; about 10.0 mm; about 15.0 mm; or about 20.0 mm. The other dimensions of the cancellous bone section 120, such as the length L_{can} or width W_{can} can be equal to or greater than about 0.5 mm; about 1.0 mm; about 1.5 mm; about 2.0 mm; about 2.5 mm; about 3.0 mm; about 3.5 mm; or about 5.0 mm.

In certain embodiments, such as the one shown in FIG. 1A, the thickness T_{can} of the cancellous bone section 120 is greater than the thickness T_{cor} of the cortical bone layer 110. The thickness T_{can} of the cancellous bone section 120 can be at least two times, about three times, about four times, about five times, about six times, about seven times, about eight times, about nine times, about ten times, about twenty times, or about fifty times greater than the thickness T_{cor} of the cortical bone layer 110. In other embodiments, the thickness T_{cor} of the cortical bone layer 110 can be equal to or greater than the thickness T_{can} of the cancellous bone section 120.

Also, in some embodiments, the width W_{cor} of the cortical bone layer 110 can be greater than the width W_{can} of the cancellous bone section 120 and/or the length L_{cor} of the cortical bone layer 110 can be greater than the length L_{can} of the cancellous bone section 120. For example, the width W_{cor} or length L_{cor} of the cortical bone layer 110 can be at least two times, about three times, about four times, about five times, about six times, about seven times, about eight times, about nine times, about ten times, about twenty times, or about fifty times greater than the width W_{can} or length L_{can} of the cancellous bone section 120

The implant shown in FIG. 1A has the shape of a three-dimensional bandage. In this embodiment, both the cortical bone layer 110 and the cancellous bone section 120 have the shape of a rectangular prism. As discussed herein, the cortical bone layer and the cancellous bone section can have various shapes and dimensions. Also, in the embodiment shown in FIG. 1A, the cancellous bone section 120 is disposed at or proximate the center of the cortical bone layer 110. In other embodiments, the cancellous bone layer may be disposed at or proximate positions other than the center of the cortical bone layer. For instance, the cancellous bone section can be disposed proximate an edge of the cortical bone layer. Moreover, in the embodiment shown in FIG. 1A, the cortical bone layer 110 is shown as being flat. In other embodiments, the cortical bone layer can be curved to varying degrees.

Furthermore, cortical bone contains collagen fibers. The cortical bone layer 110 shown in FIG. 1A, comprises a plurality of collagen fibers 130. The collagen fibers 130, in this embodiment, are oriented along the length L_{cor} of the cortical bone layer 110. In other embodiments, the collagen fibers 130 can be oriented along the width W_{cor} of the cortical bone layer 110 or along other directions.

In another embodiment, the implant suitable for delivery to a subject, such as a subject having a defect in a body part or body tissue, comprises a cancellous bone section in contact with two cortical bone layers. FIG. 1B shows a perspective view of one such embodiment. Specifically, FIG. 1B shows an implant 100a that comprises a cancellous bone section 120 (i.e., a plug section) and two cortical bone layers 110a (i.e., a cap member) and 110b (i.e., a cap member) that are in contact with the cancellous bone section 120.

The cancellous bone section 120 has a top 123 and a bottom 125. The cortical bone layer 110a has a top 113a and a bottom 115a. The cortical bone layer 110b has a top 113b and a bottom 115b. In this embodiment, the top 123 of the cancellous bone section 120 contacts the second cortical bone layer 110b and the bottom 125 of the cancellous bone section 120 contacts the first cortical bone layer 110a. Also in this embodiment, the bottom 115b of the cortical bone layer 110b contacts the cancellous bone section 120, and the top 113a of the cortical bone layer 110a contacts the cancellous bone section 120. In this embodiment, the two cortical bone layers 110a and 110b are substantially parallel to each other. The cancellous bone section 120 can be integral with the two cortical bone layers 110a and 110b.

As shown in FIG. 1B, the two cortical bone layers 110a and 100b each have a thickness or height T_{cor}, a dimension that is perpendicular to the thickness, such as the length L_{cor}, and another dimension that is perpendicular to the thickness, such as the width W_{cor}. The cancellous bone section 120 has a thickness or height T_{can}, a dimension that is perpendicular to the thickness of the cancellous bone section, such as the length L_{can}, and a another dimension that is perpendicular to the thickness of the cancellous bone section, such as the width W_{can}.

In certain embodiments, such as the one shown in FIG. 1B, the thickness T_{can} of the cancellous bone section 120 is greater than the thickness T_{cor} of the cortical bone layers 110a and 110b. In other embodiments, the thickness T_{cor} of the cortical bone layers 110a and 110b can be equal to or greater than the thickness T_{can} of the cancellous bone section 120.

Also, in some embodiments, the width W_{cor} of the cortical bone layers 110a and 110b can be greater than the width W_{can} of the cancellous bone section 120 and/or the length L_{cor} of the cortical bone layers 110a and 110b can be greater than the length L_{can} of the cancellous bone section 120.

The implant shown in FIG. 1B has the shape of an H-shaped three-dimensional bandage. In this embodiment, both the cortical bone layers 110a and 110b and the cancellous bone section 120 have the shape of a rectangular prism. As discussed herein, the cortical bone layers and the cancellous bone section can have various shapes and dimensions. Also, in the embodiment shown in FIG. 1B, the cancellous bone section 120 is disposed at or proximate the center of the cortical bone layers 110a and 110b. In other embodiments, the cancellous bone section may be disposed at or proximate positions other than the center of the cortical bone layers. For instance, the cancellous bone section can be disposed proximate an edge of the cortical bone layers. Moreover, in the embodiment shown in FIG. 1B, the cortical bone layers 110a and 110b are shown as being flat. In other embodiments, the cortical bone layers can be curved to varying degrees.

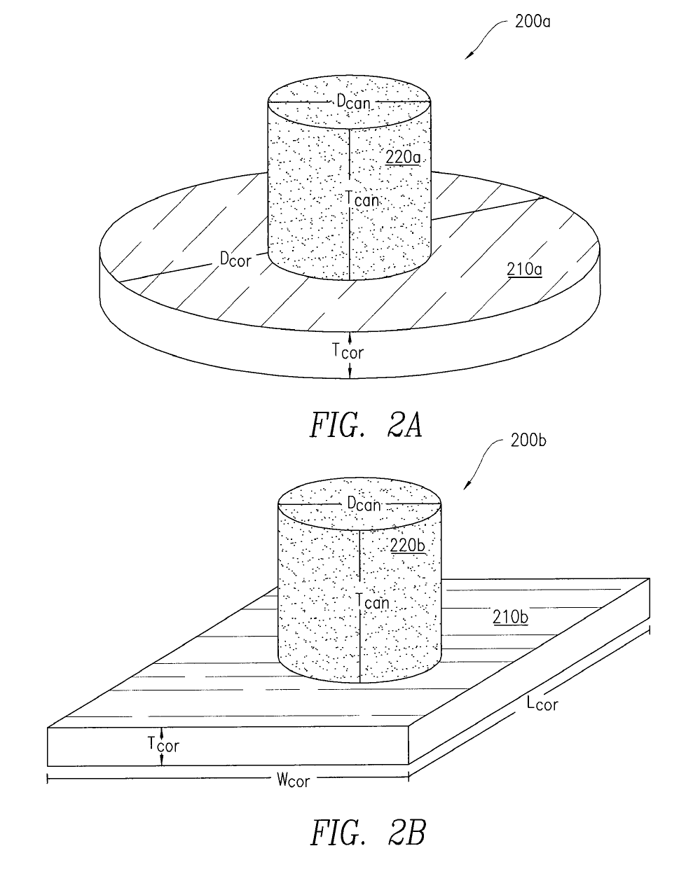

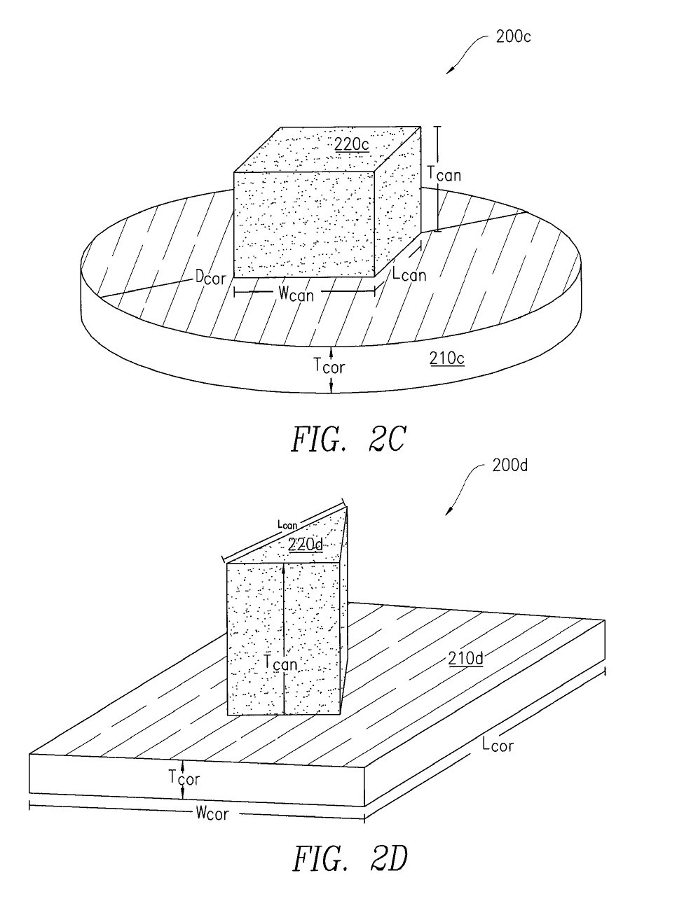

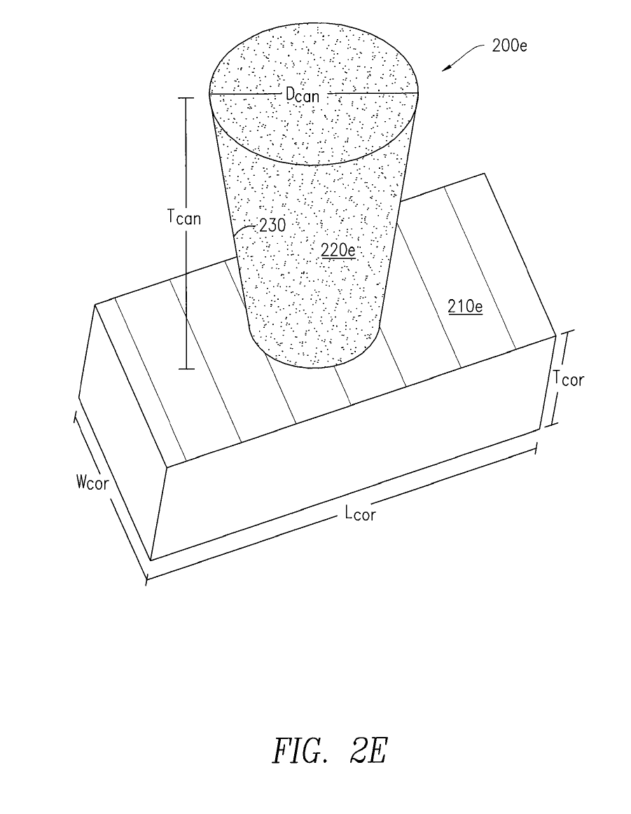

FIGS. 2A-2E show embodiments of the implants in which the cortical bone layer and cancellous bone section have various shapes. The implants can have shapes and dimensions in addition to those shown herein. Also, as shown in FIGS. 2A and 2B, the cortical bone layer and the cancellous bone section can have the same or different shapes as one another. In the implant 200a shown in FIG. 2A, the cortical bone layer 210a (i.e., a cap member) is in the shape of a cylinder and the cancellous bone section 220a (i.e., a plug section) is also in the shape of a cylinder. The cortical bone layer 210a has a thickness T_{cor} and a diameter D_{cor}. The cancellous bone section 220a has a thickness T_{can} and a diameter D_{can}. The implant 200b shown in FIG. 2B has a cortical bone layer 210b (i.e., a cap member) in the shape of a square prism and a cancellous bone section 220b (i.e., a plug section) in the shape of a cylinder. The cortical bone layer 210b has a thickness T_{cor}, length L_{cor} and width W_{cor}, which is equal in magnitude to the length L_{cor}. The cancellous bone section 220b has a thickness T_{can} and a diameter D_{can}.

FIG. 2C shows an implant 200c having a cortical bone layer 210c (i.e., a cap member) in the shape of an ovular cylinder and a cancellous bone section 220c (i.e., a plug section) in the shape of a cube. The cortical bone layer 210c has a thickness T_{cor} and a diameter D_{cor}. The cancellous bone section 220c has a thickness T_{can}, length L_{can} and width W_{can}, which are equal in magnitude.

FIG. 2D depicts another embodiment of an implant. This implant 200d has a cortical bone layer 210d (i.e., a cap member) in the shape of a rectangular prism having a thickness T_{cor}, length L_{cor} and width W_{cor}. The implant also has a cancellous bone section 220d (i.e., a plug section) in the shape of a triangular prism having a thickness Tcan and in which a side of a triangle of the prism has a length of L_{can}.

Another embodiment of an implant is shown in FIG. 2E. In this implant 200e, the cortical bone layer 210e (i.e., a cap member) is in the shape of a rectangular prism. The cortical bone layer 210e has a thickness T_{cor}, length L_{cor} and width W_{cor}. The implant 200e also comprises a cancellous bone section 220e (i.e., a plug section) that is in contact with the cortical bone layer 210e. The cancellous bone section 220e is in the shape of a cylinder with a slanted wall 230 and has a diameter D_{can} and a thickness T_{can}.

Although the embodiments in FIGS. 2A-2E are shown as having 1 cortical bone layer, they can have two cortical bone layers, like the embodiment shown in FIG. 1B. As in FIGS. 1A-1B, the cortical bone layers and cancellous bone sections of the implants depicted in FIGS. 2A-2F each have a top and a bottom. In the embodiments of FIGS. 2A-2F, the bottom of each of the cancellous bone sections contacts the cortical bone layer of the respective implant. The top of each of the cortical bone layers in the embodiments shown in FIGS. 2A-2F contacts the cancellous bone section of the respective implant. In alternative embodiments, the top of each of the cancellous bone sections contacts the cortical bone layer of the respective implant. Also in alternative embodiments, the bottom of each of the cortical bone layers contacts the cancellous bone section of the respective implant.

The bone used to make the implants described herein can be obtained from various sources. In some embodiments, the bone is obtained from a mammal, such as a human. The bone can be obtained from various parts of a mammal, such as the ilium, scapula, femur, tibia, humerus, talus, calcaneus, or patella by cutting or milling. Also, the bone used to make the implants described herein can be corticocancellous bone, cortical bone, or cancellous bone. Furthermore, the bone can be demineralized and/or rendered non-osteoinductive. Alternatively, the bone may have reduced osteoinductivity or may be treated to reduce its osteoinductivity.

In some embodiments, the implants described herein can comprise additional constituents, such as therapeutic agents. These therapeutic agents can include drugs, chemical or pharmaceutical compounds, and/or biological compounds. For instance, the implants can include various types of cells, e.g. autologous cells, allogeneic cells, nucleus pulposus cells, stem cells, progenitor cells, mesenchymal cells, stromal cells, MAPCs, MIAMI cells, chondrocytes or NP cells. Also, the implants described herein can include growth factors such as FGF, FGF 7, tGFβ and BMP, as well as bioactive molecules or cells that may accelerate tissue repair or regeneration.

In some embodiments, the implants described herein have a certain suture pull strength. The suture pull strength is the amount of force required to remove a suture used to attach the implant to body tissue. The suture pull strength can range, for example, from about 0.1 to about 4.5 MPa; about 0.45 to about 3.4 MPa; about 0.7 to about 2.5 MPa; or about 0.9 to about 1.7 MPa.

Moreover, in some embodiments, the implants described herein have a certain tensile strength. The tensile strength of the implant is the maximum stress the implant can withstand when subjected to tension, compression, or shearing. The tensile strength of the implant can range, for example, from about 1.0 to about 15.0 MPa; about 1.5 to about 12.6 MPa; about 2.5 to about 8.0 MPa; or about 3.1 to about 6.3 MPa.

The mechanical properties of the implants described herein, such as suture pull strength and tensile strength, may be linked to the preparation of the bone used to construct the implants. In particular, modifying the collagen fiber orientation of the starting cortical and cancellous tissue may strategically modify the mechanical properties of the resulting implant.

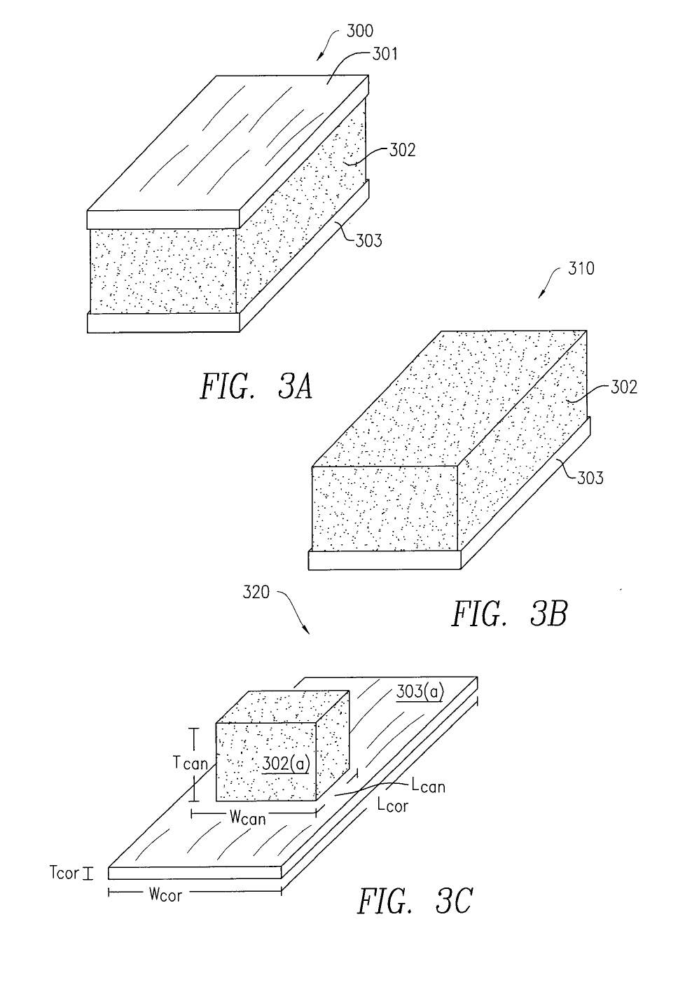

FIGS. 3A-3C show one embodiment of a method of making an implant suitable for delivery to a subject, such as a subject having a defect in a body part or body tissue. As shown in FIG. 3A, the implant may be fabricated into the desired shape from a block of corticocancellous bone 300 cut or milled from, for example, an ilium, scapula, femur, tibia, humerus, calcaneus, or patella. The bone used to make the implant may be obtained from a mammal, such as a human. In one embodiment for constructing the implant, a piece of corticocancellous bone is first cut with a cutting implement such as, but not limited to, a bandsaw, a blade, a scalpel, or a knife into a rectangular block 300 as is seen in FIG. 3A. The cortical bone regions 301, 303 are in the form of layers on top and bottom of the thicker cancellous bone layer 302. Cortical bone region 301 is integral with cancellous bone layer 302, which is also integral with cortical bone region 303.

FIG. 3B depicts the corticocancellous bone piece after removal of one of the cortical bone regions 301, which in this case is the top cortical bone region 301 shown in FIG. 3A, to produce a double-layered block 310 of corticocancellous bone. The top cortical bone region 301 may be removed by cutting with a cutting implement such as, but not limited to, a scalpel, a knife, a blade, or a bandsaw. The resultant corticocancellous block 310 has a thick cancellous bone layer 302 on top that is integral with the thinner cortical bone region 303 on the bottom.

After removal of the cortical bone region 301, the cancellous bone layer 302 and remaining cortical bone region 303 are shaped to form the implant 320, as depicted in FIG. 3C. In this Figure, the cancellous bone layer 302 is cut or milled to form the cancellous bone section 302a (i.e., a plug section) of the implant. The cortical bone region 303 is milled or cut to form a cortical bone layer 303a (i.e., a cap member), which is greater in length and width than the cancellous bone section 302a. The cancellous bone section 302a will be disposed in the center of the cortical bone layer 303a in this embodiment. As shown in FIG. 3C, the cortical bone layer 303a has a thickness or height T_{cor}, a dimension that is perpendicular to the thickness, such as the length L_{cor}, and another dimension that is perpendicular to the thickness, such as the width W_{cor}. The cancellous bone section 302a also has a thickness or height T_{can}, a dimension that is perpendicular to the thickness of the cancellous bone section, such as the length L_{can}, and a another dimension that is perpendicular to the thickness of the cancellous bone section, such as the width W_{can}. The cortical bone layer 303a is a thinner layer that is larger in surface area than that of the cancellous bone section 302a of the implant. The cancellous bone section 302a conversely is thicker than the cortical bone layer 303a.

In other embodiments of a method of making an implant suitable for delivery to a subject, such as a subject having a defect in a body part or body tissue, the cortical bone region 301 as shown in FIG. 3A is not removed. The cancellous bone layer 302 and both cortical bone regions 301 and 303 are shaped to form an implant such as 100a, as depicted in FIG. 1B. In this embodiment, the cancellous bone layer 302 is cut or milled to form the cancellous bone section 120 of the implant. The cortical bone regions 301 and 303 are milled or cut to form the cortical bone layers 110a and 110b, which are greater in length and width than the cancellous bone section. The cancellous bone section 120 will be disposed in the center of the cortical bone layers 110a and 110b in this embodiment.

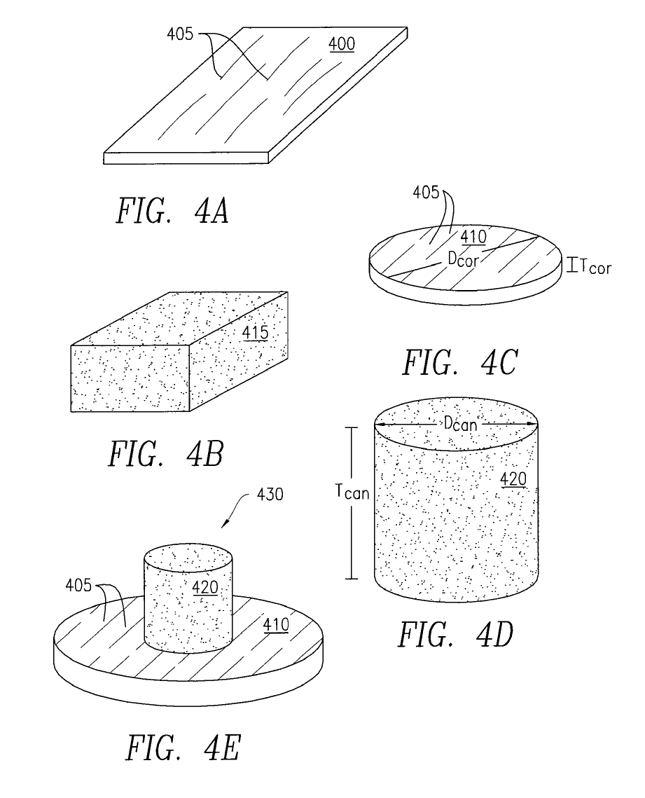

Alternatively, in some embodiments, the cortical bone layer and the cancellous bone section of the implant can be formed separately and then affixed to each other. FIGS. 4A-4E show such an embodiment of a method for making an implant in which a first portion of cortical bone and a second portion of cancellous bone are obtained separately. The portions of cortical and cancellous bone may be obtained by cutting or stripping away with a cutting implement such as, but not limited to, a scalpel, knife, blade, or bandsaw at the corticocancellous block 300 depicted in FIG. 3A. The portions of cortical and cancellous bone may instead be milled directly from an ilium, scapula, femur, or tibia of a donor, such as a mammal. FIG. 4A shows a portion of cortical bone 400 having a plurality of collagen fibers 405. FIG. 4B shows a portion of cancellous bone 415.

The portion of cortical bone 400 may be shaped using a shaping implement such as, but not limited to, a scalpel, blade, knife, or bandsaw in order to form a cortical bone layer having a first thickness and a first dimension perpendicular to the first thickness. The portion of cancellous bone 415 may be shaped using a shaping implement such as, but not limited to, a scalpel, blade, knife, or bandsaw in order to form a cancellous bone section having a second thickness and a second dimension perpendicular to the second thickness. FIG. 4C shows a cortical bone layer 410 having a plurality of collagen fibers 405 that has been shaped from a cortical bone section 400 into a cylinder having a thickness T_{cor} and diameter D_{cor}. FIG. 4D shows a cancellous bone section 420 that has been shaped from a cancellous bone portion 415 into a cylinder having a thickness T_{can} and diameter D_{can}. In order to form the implant, the cancellous bone section may be attached to the cortical bone layer such that the cancellous bone section is disposed or sitting on top of the cortical bone layer. FIG. 4E shows an implant 430 comprising a cancellous bone section 420 (i.e., a plug section) attached or affixed to a cortical bone layer 410 (i.e., a cap member). The cancellous bone section 420 and cortical bone layer 410 can be attached to each other by the use of adhesives (e.g., glues or cements, biological or otherwise), pins or dovetails.

In other embodiments, in order to form the implant, the separately formed cancellous bone section 415 or 420 may be attached to two separately formed cortical bone layers such that the cancellous bone section is disposed or sitting on top of one cortical bone layer while the second cortical bone layer is sitting on top of the cancellous bone section. FIG. 1B shows such an implant 100a comprising a cancellous bone section 120 attached or affixed to a cortical bone layer 110a on one side and attached or affixed to a second cortical bone layer 110b on the opposite side. The cancellous bone section and cortical bone layer can be attached to each other by the use of adhesives (e.g., glues or cements, biological or otherwise), pins or dovetails.

In some embodiments, the bone may be at least partially demineralized. For example, in the embodiment shown in FIGS. 3A-3C, the bone may be demineralized before or after the second cortical layer 301 of the corticocancellous bone is removed and/or before or after the corticocancellous bone is shaped into the implant. In the embodiment shown in FIGS. 4A-4E, the demineralization of the bone can occur before or after any of the steps shown. For example, the cortical bone portion 400 and cancellous bone portion 415 can be demineralized before they are shaped. Either the cortical bone portion 400 or the cancellous bone portion 415 may be demineralized, or both the cortical bone portion 400 and the cancellous bone portion 415 may be demineralized. Demineralizing can be used to form any of the implants described herein. The demineralization can be conducted using methods known to the skilled artisan. The bone may be partially demineralized, surface demineralized, or fully demineralized.

The bone may also be treated to eliminate or significantly reduce its level of osteoinductivity. The bone can be treated any time during the method of making the implant. For example, the corticocancellous bone can be treated after the first cortical region has been removed. The bone may be treated with heat to render it non-osteoinductive or to reduce its osteoinductivity. In one embodiment, the bone is treated with heat at about 50° C. for at least one hour. In another embodiment, the bone is treated with heat at about 50° C. for less than one hour. In another embodiment, the bone is treated with heat at greater than 50° C. The bone may also be treated with radiation to render it non-osteoinductive or to reduce its osteoinductivity. In one embodiment, the bone is treated with radiation to deliver a dosage of at least 20 MFrd. In another embodiment, the bone is treated with radiation to deliver a dosage of less than 20 MFrd. Alternatively, the bone may be chemically processed or treated chemically to render it non-osteoinductive or to reduce its osteoinductivity.

The implant or a portion thereof, such as the cancellous bone section, may also be compressed or compacted in a three dimensional mold or vice to a small fraction of its volume. For instance, the cancellous bone section of the implant can be compressed to have shape memory and is configured to allow extensive short-term deformation without permanent deformation, cracks, tears or other breakage in the implant. In some embodiments, following compaction, the implant or portion thereof is freeze-dried or lyophilized.

The implants described herein may be delivered to a defect or defects in a body part(s) or tissue(s) of a subject, such as a mammal, e.g., a human. The defect may be present in the intervertebral or spinal disc of the subject, the meniscus of the subject, or the cartilage, the fibrocartilage, or bone of the subject.

In certain embodiments, the implant is an intervertebral disc implant that may fully or partially replace the disc itself, or may fully or partially replace the natural or native nucleus pulposus. The implant can be configured to resist expulsion or other migration through a defect, or other opening, in the annulus fibrosis and/or to resist excessive migration within an intervertebral disc space.

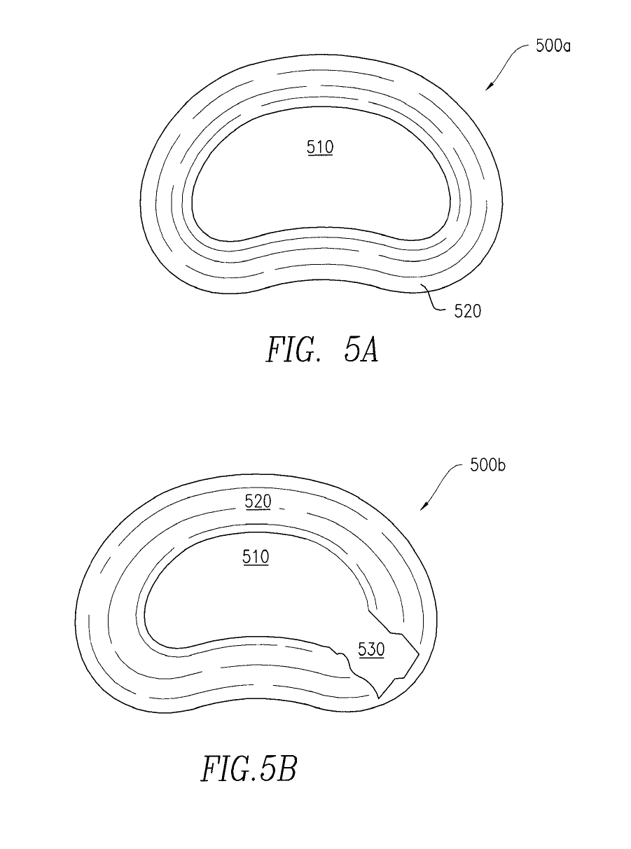

FIG. 5A shows a cross-sectional view of an intervertebral or spinal disc 500a that comprises two parts. In particular, the intervertebral disc 500a comprises an outer ring-like portion, the annulus fibrosus 520, that surrounds the nucleus pulposus 510. FIG. 5B shows an intervertebral disc 500b in which the annulus fibrosus 520 has become weakened (e.g., herniated) or defective at position 530. Because of the weakness or defect in the annulus fibrosus 520, the nucleus pulposus 510 has migrated into the annulus fibrosus 520 at position 530.

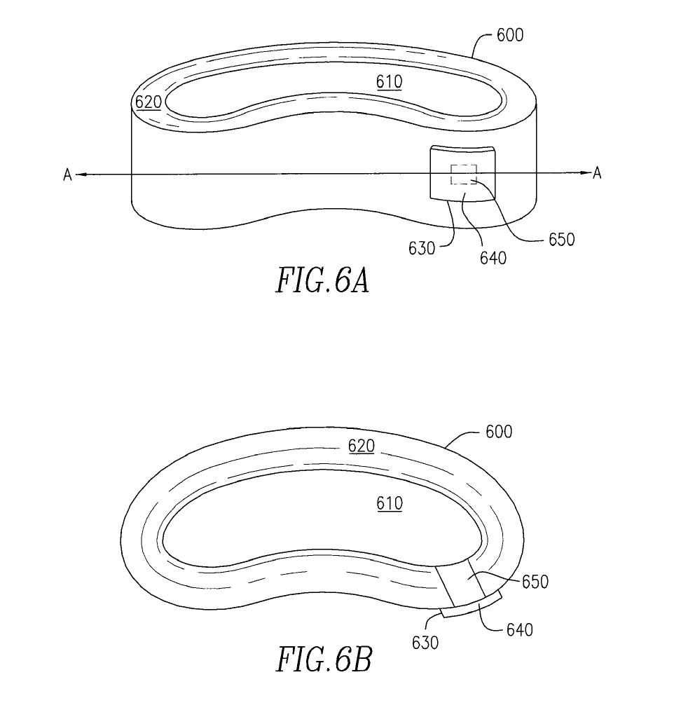

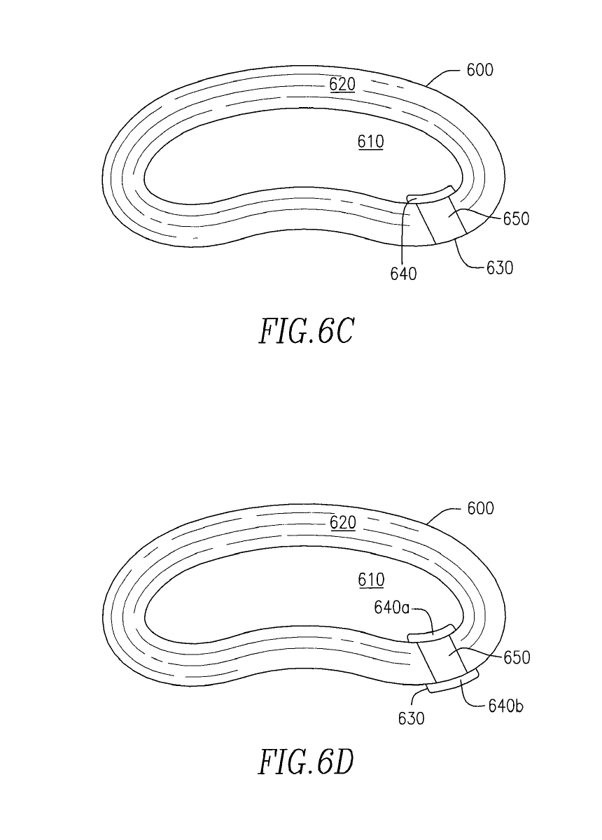

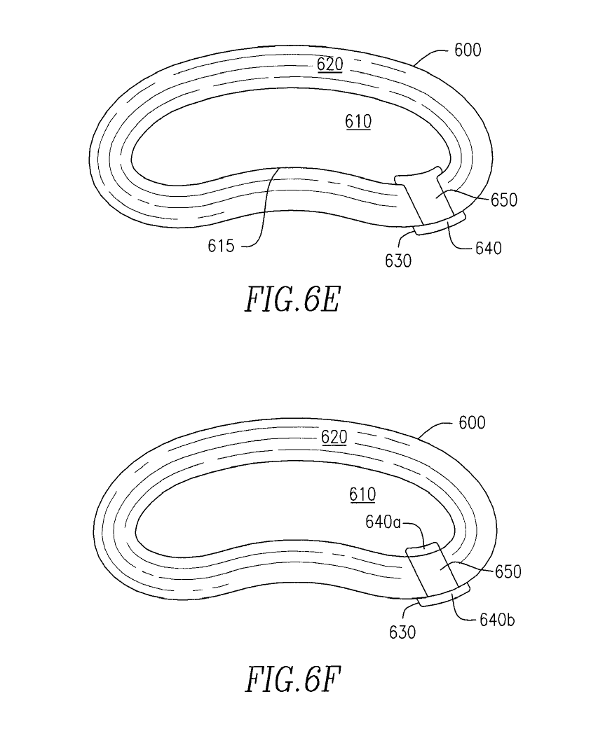

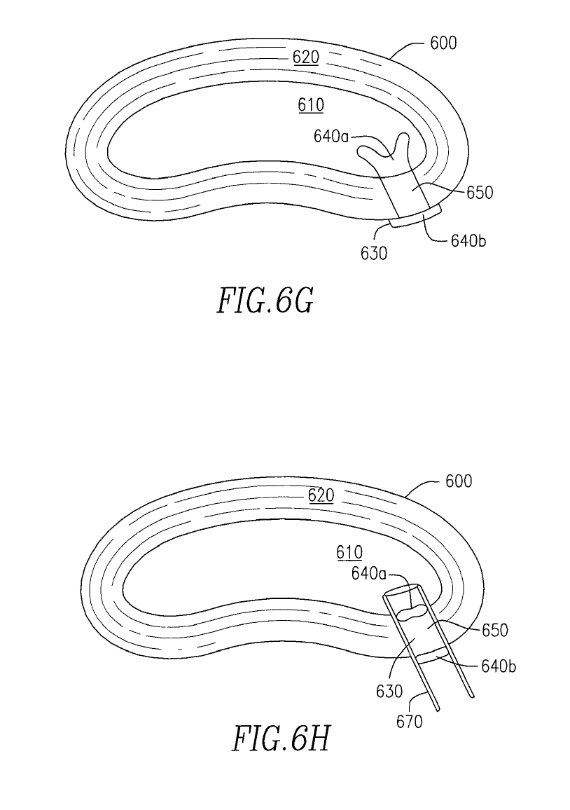

In certain embodiments, the implant can be used to repair the weakness or defect in the annulus fibrosus. FIG. 6A shows an implant 630 that has been inserted or implanted into an intervertebral disc 600, which has an annulus fibrosus 620 and nucleus pulposus 610. The implant 630 comprises a cortical bone layer 640 (i.e., a cap member) and a cancellous bone section 650 (i.e., a plug section). FIG. 6B shows a cross-section of the intervertebral disc 600 shown in FIG. 6A along the line A-A. As shown in FIG. 6B, the implant 630 has been inserted into an opening in the annulus fibrosus 620. The thickness of the cancellous bone section 650 of the implant 630 extends the full thickness of the annulus fibrosus 620. In alternative embodiments, the thickness of the cancellous bone section of the implant can extend partially through the thickness of the annulus fibrosus.

In the embodiment shown in FIG. 6B, the implant 630 is used to contain the nucleus pulposus 610 to the nucleus pulposus cavity of the intervertebral disc or to prevent the nucleus pulposus 610 from migrating into the annulus fibrosus 620. The implant 630 can be affixed to the intervertebral disc 600 by affixing the cortical bone layer 640 of the implant 630 to the outside wall of the annulus fibrosis 620. In the embodiment shown in FIG. 6C, the implant 630 is also used to contain the nucleus pulposus 610 to the nucleus pulposus cavity of the intervertebral disc or to prevent the nucleus pulposus 610 from migrating into the annulus fibrosus 620. The implant 630 can be affixed to the intervertebral disc 600 by affixing the cortical bone layer 640 of the implant 630 to the inside wall of the annulus fibrosis 620. In either of FIGS. 6B and 6C, the cortical bone layer 640 (i.e., a cap member) can, for example, be affixed to the annulus fibrosus 620 by methods such as, e.g., suturing, stapling or using adhesives, such as biological glue. In some embodiments, the implant 630 may already have sutures in it so that a surgeon implanting it would not have to thread the sutures in his or herself. In other embodiments, no additional means of attachment will be necessary. For example, in one embodiment, the shape of the implant 630 itself may act as an anchor. Furthermore, in some embodiments, the implant 630 can be entirely or partially compacted and/or lyophilized prior to or during implantation. For instance, the cancellous bone section 650 (i.e., a plug section) of the implant 630 can be compacted and/or lyophilized. In such embodiments, after the implant 630 is inserted or positioned in the intervertebral disc 600, the implant 630 or a portion thereof is uncompacted and/or hydrated. When implanted, the implant 630 or portion of the implant will expand or swell up to fill the opening or defect in the annulus fibrosus 620.

In yet another embodiment, such as the one shown in FIG. 6D, the implant 630 is used to contain the nucleus pulposus 610 to the nucleus pulposus cavity of the intervertebral disc or to prevent the nucleus pulposus 610 from migrating into the annulus fibrosus 620. The implant 630 can be affixed to the intervertebral disc 600 by affixing the cortical bone layers 640a and 640b (i.e., cap members) of the implant 630 to the inside and outside walls, respectively, of the annulus fibrosis 620. For example, the cortical bone layers 640a and 640b can, for example, be affixed to the annulus fibrosus 620 by methods such as, e.g., suturing, stapling or using adhesives, such as biological glue. In some embodiments, the implant 630 may already have sutures in it so that a surgeon implanting it would not have to thread the sutures in his or herself. In other embodiments, no additional means of attachment will be necessary. For example, in one embodiment, the shape of the implant 630 itself may act as an anchor. Furthermore, in some embodiments, the implant 630 can be entirely or partially compacted and/or lyophilized prior to or during implantation. For instance, the cancellous bone section 650 of the implant 630 can be compacted and/or lyophilized. In such embodiments, after the implant 630 is inserted or positioned in the intervertebral disc 600, the implant 630 or a portion thereof is uncompacted and/or hydrated. When implanted, the implant 630 or portion of the implant will expand or swell up to fill the opening or defect in the annulus fibrosus 620.

In further embodiments, such as the one shown in FIG. 6E, the implant 630 can be used to replace at least a portion of the nucleus pulposus 610. In this embodiment, the cancellous bone section 650 of the implant 630 extends into the nucleus pulposus cavity 615 and replaces a portion of the nucleus pulposus 610. In certain embodiments, the cancellous bone section can be folded into any shape or configuration and inserted through the annulus fibrosus and into the nucleus pulposus cavity. The cancellous bone section can then be allowed to expand or “pop” open to replace at least a portion of the nucleus pulposus.

In other embodiments, such as the one shown in FIG. 6F, the implant 630 is used to contain the nucleus pulposus 610 to the nucleus pulposus cavity of the intervertebral disc or to prevent the nucleus pulposus 610 from migrating into the annulus fibrosus 620. The implant 630 can be affixed to the intervertebral disc 600 by affixing the cortical bone layers 640a and 640b of the implant 630 to the inside and outside walls, respectively, of the annulus fibrosis 620. Cortical bone layer 640a may be compacted and/or lyophilized prior to and during implantation as shown in FIG. 6F. After implantation, cortical bone layer 640a may then be or become uncompacted and/or rehydrated such that it resembles cortical bone layer 640a in FIG. 6D. The principle of compacting and/or lyophilizing the cortical bone layer prior to and during implantation and then uncompacting or rehydrating the cortical bone layer(s) after implantation as shown in FIG. 6F may also apply to other embodiments, including the embodiments shown in FIGS. 6C and 6E.

FIG. 6G shows another embodiment, in which the implant 630 is used to contain the nucleus pulposus 610 to the nucleus pulposus cavity of the intervertebral disc or to prevent the nucleus pulposus 610 from migrating into the annulus fibrosus 620. The implant 630 can be affixed to the intervertebral disc 600 by affixing the cortical bone layers 640a and 640b of the implant 630 to the inside and outside walls, respectively, of the annulus fibrosis 620. Prior to and during implantation, cortical bone layer 640a may be bent and lyophilized such that there are two flaps extending into the nucleus pulposus cavity of the intervertebral disc, as shown in FIG. 6G. After implantation, cortical bone layer 640a may then be or become uncompacted and/or rehydrated such that it resembles cortical bone layer 640a in FIG. 6D. The principle of compacting and/or lyophilizing the cortical bone layer prior to and during implantation and then uncompacting or rehydrating the cortical bone layer(s) after implantation as shown in FIG. 6G may also apply to other embodiments, including the embodiments shown in FIGS. 6C and 6E.

FIG. 6H depicts a shear 670 which holds an implant 630 in a collapsed or compacted or lyophilized position for insertion through an opening in the annulus fibrosus 620 of an intervertebral disc 600. Once inserted, the implant 630 can be affixed to the intervertebral disc 600 by affixing the cortical bone layers 640a and 640b of the implant 630 to the inside and outside walls, respectively, of the annulus fibrosis 620. After implantation, the implant 630 may then be or become uncompacted and/or rehydrated such that it resembles cortical bone layer 640a in FIG. 6D. The principle of using a shear to hold an implant in a compact or collapsed or lyophilized position for insertion through an opening in the annulus fibrosis of an intervertebral disc prior to and during implantation and then uncompacting or rehydrating the implant after implantation as shown in FIG. 6H may also apply to other embodiments, including the embodiments shown in FIGS. 6C and 6E.

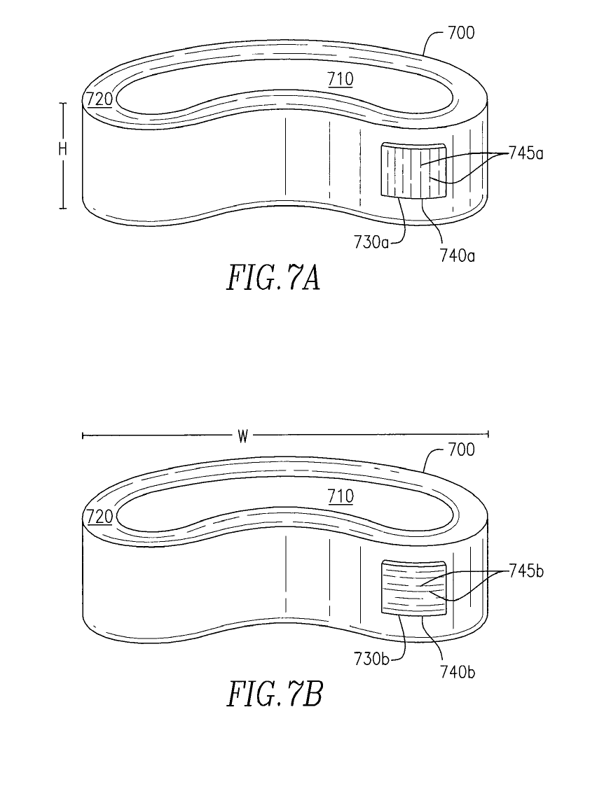

FIGS. 7A and 7B show two additional embodiments of implants 730a and 730b that have been implanted into an intervertebral disc 700 having an annulus fibrosus 720 and a nucleus pulposus 710. In FIG. 7A, the implant 730a has been positioned with respect to the intervertebral disc 700 such that the collagen fibers 745a of the cortical bone layer 740a of the implant 730a are oriented along the height H of the intervertebral disc 700. In FIG. 7B, the implant 730b has been positioned with respect to the intervertebral disc 700 such that the collagen fibers 745b of the cortical bone layer 740b of the implant 730b are oriented along the width W of the intervertebral disc 700.

In certain embodiments, the implant is a meniscal implant that may fully or partially replace the meniscus itself, or may repair rips, tears, openings, damages or defects in the native or natural meniscus. The implant can be configured to resist expulsion or other migration through a defect, or other opening, in the meniscus and/or to strengthen a defective, torn, ripped, weakened or damaged meniscus.

FIG. 8 shows a superior (top) view of a right knee 800 with a medial meniscus 810 and a lateral meniscus 820. The knee 800 may be a knee of a mammal, such as a human.

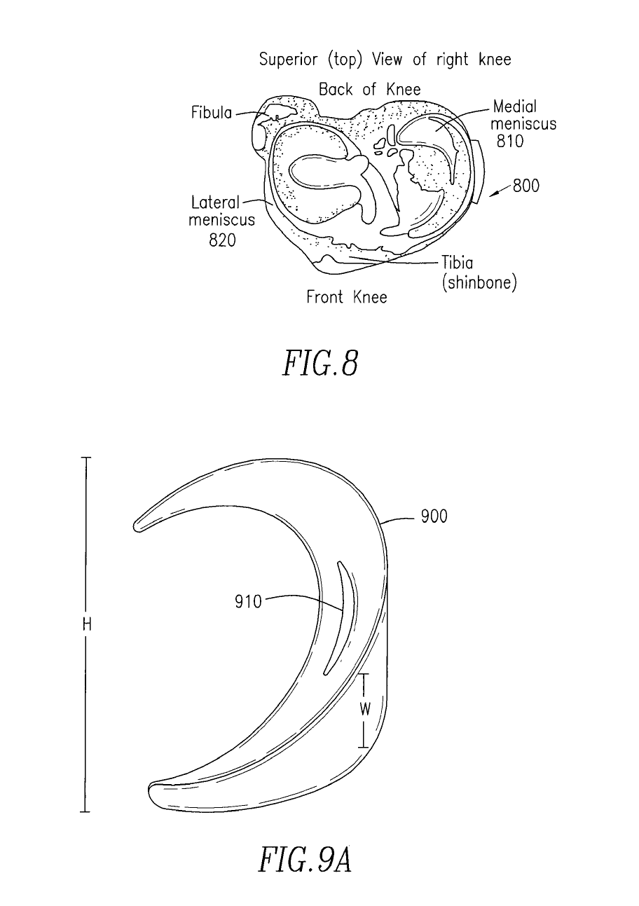

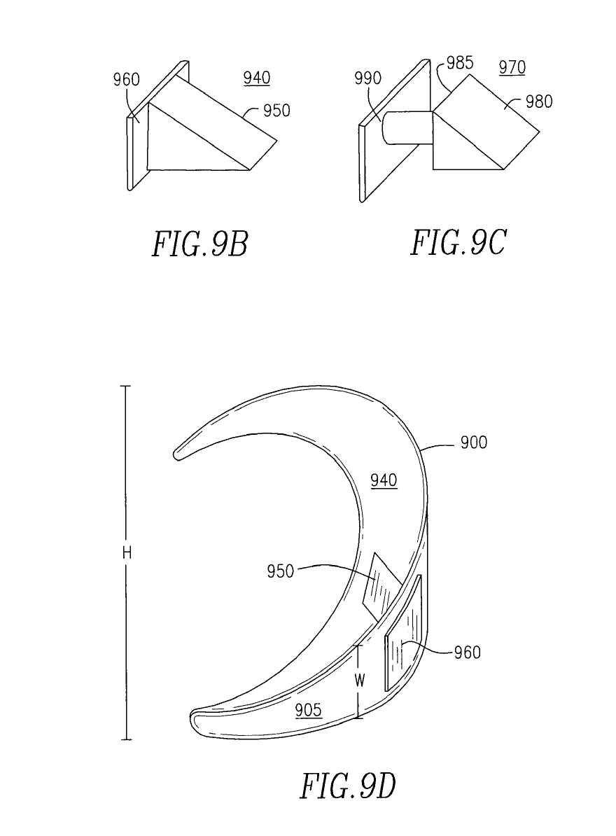

FIG. 9A depicts a meniscus 900 having a circumferential tear 910. The meniscus 900 has a height H and a width W.

The implants described herein may be shaped specifically for use in meniscal repair, as shown in FIGS. 9B and 9C. FIGS. 9B and 9C show embodiments of implants 940 and 980 for meniscal repair. The implants may be cut or milled so as to form a cortical bone layer 960 and a wedge-shaped cancellous bone section 950 (i.e., a plug section), or a cortical bone layer 990 (i.e., a cap member), a cancellous stem section 985 and a cancellous distal wedge-shaped section 980 The cancellous bone section 950 or 980 is shaped so as to mimic the natural geometry of the meniscus, and the cortical bone layer 960 or 990 is greater in length and width than the cancellous bone section 950 or 980. The cancellous bone section 950 or 980 will be disposed in the center of the cortical bone layer 960 or 990 in these embodiments. Other embodiments of an implant for use in meniscal repair may also have a second cortical bone layer such that the cancellous bone section is sitting between and is attached to two cortical bone layers.

FIG. 9D shows an embodiment of an implant 940 which has been delivered to or implanted in a meniscus 900 having a circumferential tear, as depicted in FIG. 9A. After the torn or ripped tissue has been removed from the meniscus 900 or the meniscus 900 has been partially resected, the cortical bone layer 960 of the implant 940 may be placed adjacent to the meniscal rim 905 and may be used to secure the implant 940 to the remaining meniscus 900. The cortical bone layer 960 of the implant 940 may be attached to the meniscus 900 by suturing or stapling a portion of the cortical bone layer 960 to the meniscus 900 or by using a biological glue or adhesive which adheres the cortical bone 960 layer to the meniscus 900. The cortical bone layer 960 may contain a plurality of collagen fibers, and the implant 940 may be attached to the meniscus 900 such that the collagen fibers are oriented along the height H of the meniscus. Alternatively, the implant 940 may be attached to the meniscus 900 such that the collagen fibers are oriented along the width W of the meniscus. The implant 940 may be compacted or lyophilized prior to implantation or delivery to a defect of a meniscus and may be allowed to expand subsequent to implantation or delivery to a defect of a meniscus.

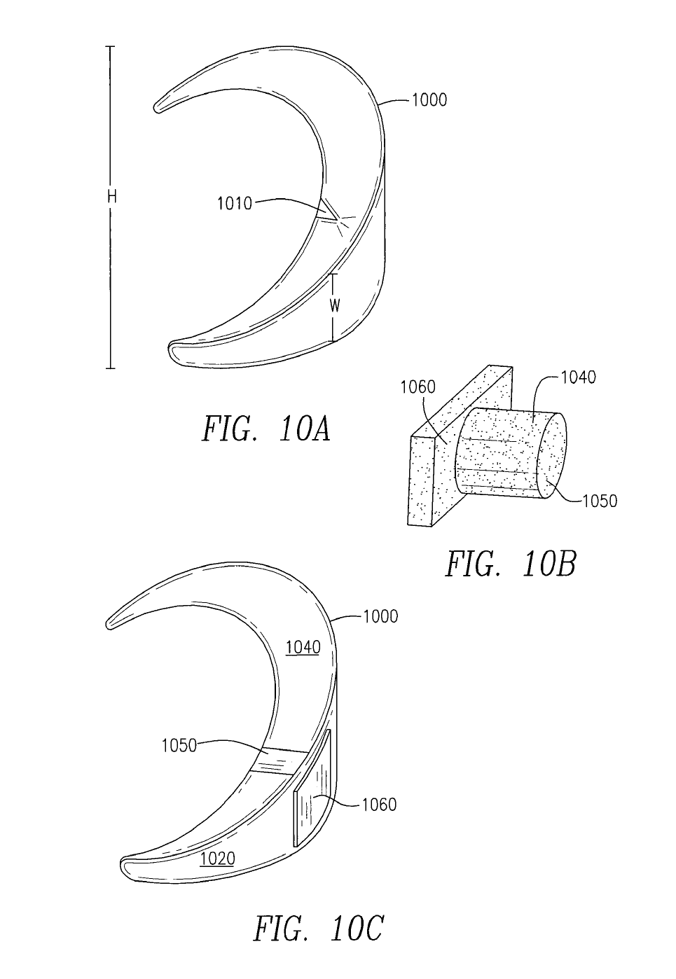

FIG. 10A depicts a meniscus 1000 having a radial tear 1010. The meniscus 1000 has a height H and a width W. FIG. 10B shows an embodiment of an implant 1040 specifically shaped for use in meniscal repair. The implant may be cut or milled so as to form a cortical bone layer 1060 (i.e., a cap member) and a cancellous bone section 1050 (i.e., a plug section) which is cylindrical, wherein the cortical bone layer 1060 is greater in length and width than the cancellous bone section 1050. The cortical bone layer 1060 may also have a conical or slanted cone shape. The cancellous bone section 1050 will be disposed in the center of the cortical bone layer 1060 in this embodiment. Other embodiments of an implant for use in meniscal repair may also have a second cortical bone layer such that the cancellous bone section is sitting between and is attached to two cortical bone layers.

FIG. 10C shows an embodiment of an implant 1040 which has been delivered to or implanted in a meniscus 1000 having a radial tear, as depicted in FIG. 10A. After the torn or ripped tissue has been removed from the meniscus 1000 or the meniscus 1000 has been partially resected, a trephine may be used to bore a channel from the outer edge of the meniscus 1020 down to the base of the tear. The cancellous portion 1050 of the implant 1040 may then be inserted into the created channel, and the cortical bone layer 1060 may be used to secure the implant 1040 to the surrounding meniscal rim 1020. The cortical bone layer 1060 of the implant 1040 may be attached to the meniscus 1000 by, e.g., suturing or stapling a portion of the cortical bone layer 1060 to the meniscus 1000 or by using a biological glue or adhesive which adheres the cortical bone 1060 layer to the meniscus 1000. The cortical bone layer 1060 may contain a plurality of collagen fibers, and the implant 1040 may be attached to the meniscus 1000 such that the collagen fibers are oriented along the height H of the meniscus. Alternatively, the implant 1040 may be attached to the meniscus 1000 such that the collagen fibers are oriented along the width W of the meniscus. The implant 1040 may be compacted or lyophilized prior to implantation or delivery to a defect of a meniscus and may be allowed to expand subsequent to implantation or delivery to a defect of a meniscus.

Any of the embodiments of the implant described herein may comprise a therapeutic agent and be used to deliver the therapeutic agent to body tissue. The therapeutic agent can be incorporated into the cancellous bone section and/or the cortical bone layer. For example, the cancellous bone section of the implant, which may be packaged in a moist configuration, can be used as a matrix to absorb the therapeutic agent. Also, the implant comprising the therapeutic agent can be frozen before use and stabilized with cryoprotectants before freezing. In addition, the therapeutic agent, such as cells obtained from a patient, can be introduced into the implant during formation of the implant or just prior to insertion of the implant into the patient. Suitable therapeutic agents include those described above.

In another embodiment, the implant could be inserted into an intervertebral disc with natural, recombinant or synthetic polymers. For example, polymers could be used to: (1) seal the annular gap by acting as a “bio-glue,” (2) provide additional load-bearing capacity (e.g., recombinant polymers are available, composed of alternating elastin and silk segments), and/or (3) act to stabilize the implant and cells to avoid extrusion of the implant or materials thereof.

In some embodiments, the implant may be used in applications other than the repair or treatment of defects in an intervertebral disc or a meniscus. For instance, the implant can be used to repair defects in the cartilage, fibrocartilage, or bone.

The description contained herein is for purposes of illustration and not for purposes of limitation. The methods and constructs described herein can comprise any feature described herein either alone or in combination with any other feature(s) described herein. Changes and modifications may be made to the embodiments of the description. Furthermore, obvious changes, modifications or variations will occur to those skilled in the art. Also, all references cited above are incorporated herein, in their entirety, for all purposes related to this disclosure.

The following illustrative examples are set forth to assist in understanding the methods and constructs described herein and do not limit the claimed methods and constructs.

### EXAMPLES

### Example 1

### Preparation of an Implant

Two sets of donor ilia (two ilia each from a 27 year old male and a 51 year old male with a respective yield of 12 implant prototypes and 10 implant prototypes) were selected for the preparation of three dimensional bandage-shaped implant prototypes to be utilized for mechanical testing in the context of annulus fibrosus repair.

The thicker regions of donor ilia were cut into 1 cm×2 cm cross-sections (size specification shown below) using a bandsaw.

| L_{CORT} | W_{CORT} | H_{CORT} | D_{CANC} | H_{CANC} | V_{OFFSET} | H_{OFFSET} |
|---|---|---|---|---|---|---|
| (mm) | (mm) | (mm) | (mm) | (mm) | (mm) | (mm) |
| 20 | 10 | 10 | 8 | 5 | 6 | 1 |

Each of the samples contained a relatively thick cancellous layer (>5 mm) sandwiched between two thinner cortical layers. The tissue was cut so that the collagen fibers were oriented either lengthwise or widthwise and the orientation of the collagen fiber was noted. The cut tissue samples were subsequently processed and demineralized.

Next, using a scalpel, one of the thin cortical layers from each sample was stripped off with caution to not cut or damage the tissue. The removed cortical strips were set aside for tensile and suture pull (i.e., mechanical) testing.

Following that step, the cancellous layer of the samples was shaped, using a scalpel, into a smaller block or cylinder-like shape having a height of approximately 5 mm and a diameter or length of approximately 8 mm. Samples that possessed poor cancellous density (i.e., web-like cancellous rather than dense cancellous) were discarded. From each ilium, 5 to 6 pieces or samples were obtained that met the criteria. These cut tissues were then packaged in kapak and stored at 4° C.

What is claimed is:

1. An implant for repair of a defective annulus fibrosus, said implant comprising:
a plug section sized and shaped so as to be insertable in a defect in an annulus fibrosus, said plug section being made from demineralized cancellous bone and having a pair of opposed ends; and
at least one cap member attached to at least one of said ends of said plug section so as to inhibit movement of said implant in said defect, said at least one cap member being made from cortical bone, wherein at least a portion of said implant is non-osteoinductive.
